(19) United States
(12) Patent Application Publication
(10) Pub. No.: US 2003/0150048 A1
(43) Pub. Date: Aug. 14, 2003

(54) Face shield that attaches to existing eye goggle

(76) Inventor: Lloyd R. Price, Fair Oaks, CA (US)

Correspondence Address: Lloyd R. Price, 8552 White Hawk Ct, Fair Oaks, CA, 95628, US

(21) Appl. No.: 10/067,907

(22) Filed: Feb. 8, 2002

(51) Int. Cl. A61F 9/02

(52) U.S. Cl. 2/431

(57) ABSTRACT

Face shield that is mounted to existing goggles commonly used in outdoor active sports such as snow sports. This face shield attaches to the securing straps of the existing goggle as a primary feature. This face shield follows the contour and shape of an existing goggle with the top edge of the face shield extending horizontally across the users forehead, past both of the temples as it wraps around the side of the face and head where it terminates at or past the ears, with the lower front of the face shield extending to or past the user's chin. This face shield creates a protected airspace in front of the users nose and mouth to allow adequate air circulation while providing excellent facial protection from wind, weather and precipitation. Furthermore, this face shield can be used in conjunction with a protective helmet and other weather protection and safety equipment commonly used in outdoor active sports.

### BACKGROUND OF THE INVENTION

### REFERENCES TO PRIOR ART

U.S. Patent Documents

U.S. Pat. No. 4,621,378 May 1984 Hatchman . . . 2/9

U.S. Pat. No. 5,138,723 August 1992 Bolle . . . 2/430

U.S. Pat. No. 5,214,804 June 1993 Carey, Edwards . . . 2/206

U.S. Pat. No. 5,704,068 January 1998 Martin . . . 2/173

In certain outdoor activities, such as snow skiing, eye goggles are used to protect the eyes and upper face from weather, wind and precipitation.

In certain outdoor activities, it is desirable to have protection from weather, wind and precipitation on the entire face including the nose mouth and cheeks.

The prior art face shields have several drawbacks.

Those face shields providing their own attachment to the users head or ears such as a headband or frame that rests on, or is tied, strapped or held on to the users head with an elastic or non elastic strap or bracket of some type cannot be used in conjunction with existing eye goggles commonly adapted for use in outdoor active sports.

Those face shields providing their own attachment to the users head or ears such as a headband or frame that rests on, or is tied, strapped or held on to the users head with an elastic or non elastic strap or bracket of some type cannot be used in conjunction with protective helmets which have been commonly adapted for use in outdoor active sports.

Those face shields that are designed to use any part of the face primarily or as part of its mounting to the user do not allow for adequate air circulation for breathing especially during outdoor active sports.

Those face shields that cover the entire face with the eyes, nose and mouth sharing the same airspace behind the face shield are prone to fogging and compromising vision in outdoor activity sports when conditions promote condensation.

### BRIEF SUMMARY OF INVENTION

There is, therefore, provided in the various embodiments of the present invention hereinafter set forth, a faceshield adapted to overcome the drawbacks noted in the background.

This faceshield is designed to be used in conjunction with an existing eye goggle that is commonly used in outdoor active sports such as snow skiing.

This faceshield is made from a semi flexible sheet plastic and is designed to conform to the shape and contour of an existing goggle used in outdoor active sports.

This faceshield covers the frontal face area that extends from the top of the forehead to the bottom of the chin, and extends in a horizontal plane to or past the users temples to the sides, and follows the average faceline vertically to the bottom of the horizontal plane of the users chin.

In addition, the faceshield wraps around the side of the user's face and head past the users temples on both sides and extends to or past the users ears, covering the side of the user's face and cheeks, following the contours and shape of the existing goggle.

This faceshield covers the frontal face area, wraps around the side of the user's face and head and extends to or past the users ears, covering the side of the user's face and cheeks; following the contours and shape of the existing goggle.

Attachment straps on the faceshield made from hook and loop or a similar material are located in a position corresponding to the existing goggle's mounting or attachment straps and is secured by said attachment straps on the faceshield wrapping around the existing goggle's attachment strap. The positioning of these straps is user adjustable based on different sizes of existing goggle available. The user chooses the position for the strap on the faceshield that corresponds to the location of the strap on their existing goggle. This manner of attachment is to be used as this faceshield's primary mounting method to the existing goggle, which are to be worn as their original manufacturer intended.

Furthermore, this faceshield has been specifically designed to work in conjunction with most brands and sizes of eye goggles and helmets currently available in the retail marketplace.

This faceshield has an opening in the center of the faceshield that corresponds to an average size of a lens found in an existing goggle. This opening is the size of an average user's field of vision and is located on the approximate centerline of the existing goggle that the faceshield is attached to which allows the user to have no limitations of vision due to using this faceshield and allows the existing goggles to be used as their original manufacturer intended. Furthermore, when attached to an existing eye goggle, this faceshield can be used without interference caused by or imposed on a protective helmet commonly used in outdoor active sports. This faceshield has been specifically designed to work in conjunction with most brands and sizes of eye goggles and helmets currently available in the retail marketplace.

### BRIEF DESCRIPTION OF DRAWINGS

The drawings contained herein this application illustrate the basic and detailed shape and design of this faceshield.

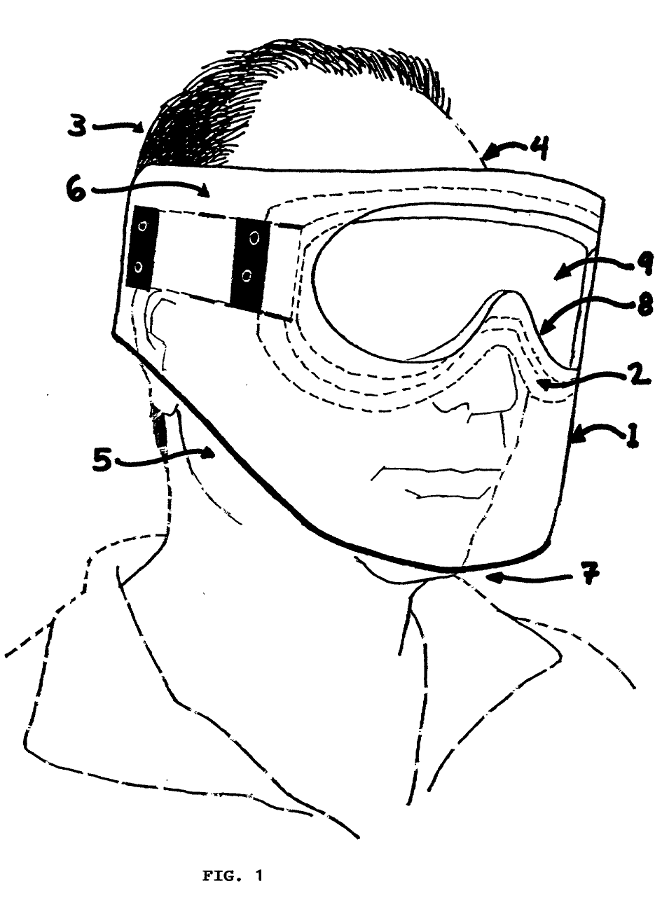

Page 1 FIG. 1 is a front view thereof

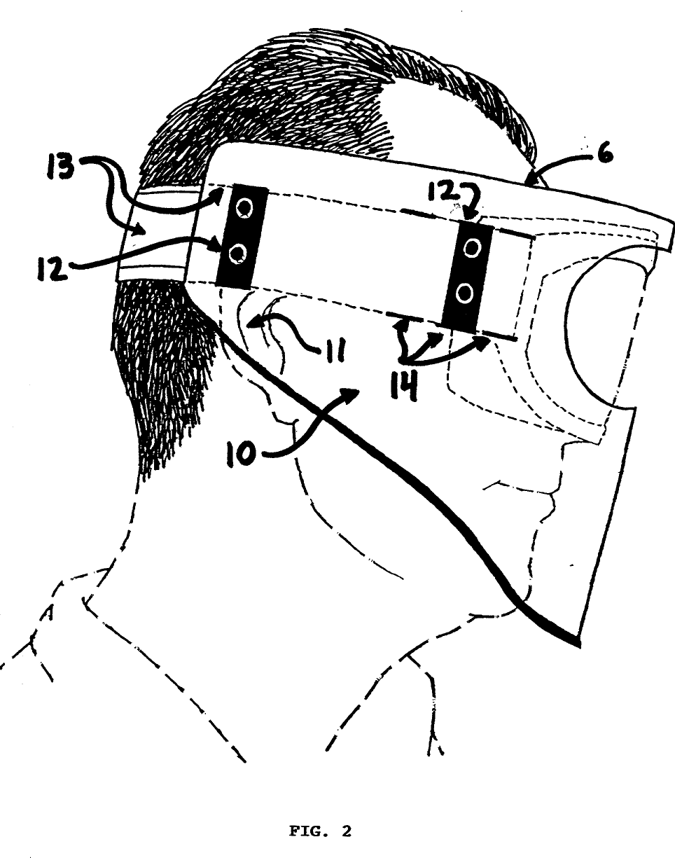

Page 2 FIG. 2 is a side view thereof

### DETAILED DESCRIPTION OF PREFERRED EMBODIMENT

The drawings contained herein illustrate the shape, design, details and features of this face shield.

In addition, these drawings illustrate the unique method of attaching this face shield to an existing eye goggle.

FIG. 1 is a front view that shows the overall design and shape of this face shield 1.

In addition FIG. 1 shows the intended mounting to an existing goggle 2 and a typical user's head 3, which are both represented by a broken outline.

This face shield 1 covers the frontal face area that extends from the forehead 4 to the bottom of the chin 5, and extends in a horizontal plane to or past the users temples 6 to the sides, and follows the average faceline vertically to the bottom of the horizontal plane of the users chin 7.

This face shield has an opening 8 in the center of the face shield that corresponds to an average size of a lens 9 found in an existing goggle 2.

This opening is the size of an average user's field of vision and is located on the approximate centerline of the lens 9 of the existing goggle 2 that the face shield 1 is attached to.

FIG. 2 is a side view that illustrates the section of the face shield that wraps around the side of the user's face and head 10 past the users temples 6 on both sides and extends to or past the users ears 11, covering the side of the user's face and cheeks.

This faceshield has attachment straps 12 on the sides of the faceshield that correspond to the orientation of the existing goggle's retention strap 13.

The faceshield's attachment straps 12 are made of hook and loop and wrap around the existing goggle strap as this faceshield's primary attachment method. These straps 12 are adjustable for different sizes of goggles by the user choosing the position 14 for the faceshield's attachment strap that corresponds to the location of their existing goggle retention strap.

Left and right views of FIG. 2 are exact mirror images of each other.

What I claim is:

1. A face shield adapted for use in outdoor active sports such as snow sports.

2. The face shield of claim 1 wherein said face shield is made from semi-flexible plastic and is formed to match the contour and overall shape of an existing eye goggle commonly used in outdoor active sports such as snow sports.
Furthermore this face shield covers the existing goggle and extends past the existing goggle to terminate at the user's forehead following a horizontal plane to or past the temples on both sides, to or past the user's chin on the bottom of the face shield, and wrapping around the side of the face and head to terminate at or past the user's ears.

3. The faceshield of claim 1 wherein said faceshield has straps made of hook and loop that are in a corresponding position to the securing straps of the existing goggles on the side of the head.
This faceshield's straps wrap around the securing strap from the existing goggle, and is the primary attachment method and feature. The faceshield's attachment straps are adjustable for the user to position the straps to correspond to different sizes of goggles available in the retail marketplace and the different respective locations of their securing straps.
This face shield's attachment method allows the existing eye goggle to be worn as their original manufacturer intended.

4. The face shield of claim 1 wherein said face shield creates an airspace between the users face and face shield when this face shield is attached to an existing goggle by the prescribed attachment method.

5. The face shield of claim 1 wherein said face shield has an opening in the frontal area that corresponds in size and shape to an existing goggle's lens. This opening allows for unobstructed vision by the user of this face shield. This face shield allows the existing goggle to be used as their original manufacturer intended.

6. The face shield of claim 1 wherein said face shield can be used in conjunction with protective helmets that are commonly used in outdoor active sports such as snow sports.

7. The face shield of claim 1 wherein said face shield can be used in conjunction with most eye goggles used in outdoor active sports that are currently available for retail sale.

8. The face shield of claim 1 wherein said face shield has a frontal surface area suitable for application of graphics, logos, proper names or other appropriate artwork or printing.
